(19) United States
(12) Patent Application Publication
(10) Pub. No.: US 2025/0161346 A1
(43) Pub. Date: May 22, 2025

(54) METHODS FOR TREATING HEPATITIS B VIRUS INFECTION

(71) Applicants: Chiaho Shih, Houston, TX (US); KAOHSIUNG MEDICAL UNIVERSITY, Kaohsiung City (TW)

(72) Inventors: Chiaho Shih, Houston, TX (US); Ching-Chun Yang, Taipei City (TW); Chih-Hsu Chang, Taoyuan City (TW)

(73) Assignees: Chiaho Shih, Houston, TX (US); KAOHSIUNG MEDICAL UNIVERSITY, Kaohsiung City, TW (TW)

(21) Appl. No.: 18/842,040

(22) PCT Filed: Mar. 6, 2023
(86) PCT No.: PCT/CN2023/063751

§ 371 Date: Aug. 27, 2024

Related U.S. Application Data
(60) Provisional application No. 63/317,084, filed on Mar. 7, 2022.

(51) Int. Cl. A61K 31/713; A61K 38/21; A61P 31/20

(52) U.S. Cl. CPC A61K 31/713; A61K 38/21; A61P 31/20

(57) ABSTRACT

A method for treating symptoms of a disease associated with an infection caused by hepatitis B virus (HBV), in particular a chronic HBV infection, is disclosed. The method includes administering a composition containing a CRM1 inhibitor into an HBV-infected subject, and the CRM1 inhibitor has the function of inhibiting a nuclear export pathway of HBV core particles and blocking HBV virion secretion.

No new matter is added.

### CROSS-REFERENCE TO RELATED APPLICATIONS

This application is a national stage application, filed under 35 U.S.C. § 371, of International Patent Application No. PCT/US2023/063751 filed on Mar. 6, 2023, which claims the benefit of U.S. Provisional Patent Application No. 63/317,084, filed Mar. 7, 2022, each of which is incorporated by reference herein in its entirety.

### FIELD OF THE INVENTION

The present invention relates to a method for treating hepatitis B virus infection by using a CRM1 inhibitor, in particular the CRM1 inhibitor is able to inhibit a nuclear export pathway of HBV core particles.

### BACKGROUND OF THE INVENTION

According to the estimate by World Health Organization (WHO) in 2019, there are more than 300 million chronic hepatitis B virus (HBV) carriers worldwide. Despite the fact that highly effective vaccines for hepatitis B virus (HBV) have been available for more than four decades, a curative therapeutic treatment for HBV remains an unmet medical need. Current clinical therapy relies on the oral intake of nucleot(s)ide analogs, such as tenofovir or entecavir. While these drugs can inhibit viral growth, they cannot completely eradicate the virus from the liver. Chronic hep B Patients need to continue the lifetime therapy with nucleot(s)ide analogs. Long term treatment invariably induced the emergence of drug-resistant variants.

CRM1 (chromosome region maintenance 1, exportin1, Xpo1) is a major receptor for the export of protein cargos out of the nucleus. SINE compounds (selective inhibitor of nuclear export) are the best known CRM1 inhibitors which had been tested for cancer treatments. In addition, SINE compounds, such as Verdinexor (KPT-335), had been tested as an antiviral for influenza virus, and respiratory syncytial virus (RSV). Another compound, Eltanexor (KPT-8602), can inhibit human cytomegalovirus (HCMV) by increasing the expression of type I interferon. KPT-8602 also reduced the lytic replication of Kaposi's sarcoma-associated herpesvirus (KSHV).

### SUMMARY OF THE INVENTION

The present invention relates to a composition for use in treating symptoms of a disease associated with an infection caused by hepatitis B virus (HBV), wherein the composition comprising a therapeutically effective amount of a CRM1 inhibitor.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A and 1B show the CRM1 machinery facilitates nuclear export of encapsidated viral RNA. FIG. 1A shows that NES^{CRM1} mutation reduces capsid-associated viral RNAs only in the cytoplasm, but not in the nucleus. HuH-7 cells are transfected with WT-HBV and NES^{CRM1} I+II mutant (mtNES^{CRM1}-I+II). Separated nuclear and cytoplasmic fractions from transfected cell lysates are analyzed using 5 different assays for encapsidated viral RNAs, total viral RNAs (encapsidated+non-encapsidated), ribosomal RNAs, capsid particles, and α-tubulin. Upper panel: Encapsidated viral RNAs are compared between nuclear and cytoplasmic fractions. Total protein concentration in each fraction is measured by the Bio-Rad protein assay. Capsid particles from the same amount (1.4 mg) of total protein per lane are PEG-precipitated. Encapsidated viral RNAs are then extracted and analyzed by Northern blot using the HBV core (HBc) probe. Middle panel: Total intracellular RNAs from nuclear and cytoplasmic compartments are compared between WT and NES mutant using the HBs probe by Northern blot. Equal amount (20 μg) of total RNA samples is loaded on each lane. Ribosomal RNAs (rRNAs) are included as loading controls. * Nuclear precursors of rRNAs. Lower panel: Capsid particles were measured by native agarose gel using anti-core antibody. Tubulin: a cytoplasmic marker. Equal amounts of total protein were loaded on each lane of agarose gel or SDS-PAGE. FIG. 1B shows that HBV polymerase is essential to pgRNA encapsidation in both cytoplasm and nucleus. The present invention compares the levels of encapsidated and total viral RNAs in the nucleus and cytoplasm between WT HBV and mutant NES^{CRM1}-I and NES^{CRM1}-II, with or without a functional polymerase (ΔPol). A similar experimental design as in FIG. 1A, is used to evaluate the role of polymerase (Pol) in pgRNA encapsidation in the nucleus. Lack of pgRNA encapsidation is rescued by cotransfection with a WT polymerase. Banding intensities are quantified by densitometry and the Image J software, and the ratios are normalized to the cytoplasmic signals of WT samples. For capsid-associated RNAs, signals of the entire lane are quantified. For total intracellular viral RNAs, only the quantified 3.5 kb pgRNA is compared. Both 28S and 18S rRNAs are included for quantification. WT: wild type. Cy: cytoplasm. Nu: nucleus.

FIGS. 2A to 2F show that early co-treatment with a CRM1 inhibitor can strongly inhibit pgRNA encapsidation and DNA synthesis in an HBV infection system. FIG. 2A shows that immunofluorescence microscopic assay (IFA) is performed to analyze the subcellular localization of HBc. The mock treatment with no drug exhibits a cytoplasm-predominant pattern of HBc (C (cytoplasm)>N (nucleus)). When HBV infection is treated with a viral entry inhibitor (myr-preS1 peptide) at 3 hrs before infection, no HBc signal is detected. However, HBc is strongly arrested in the nucleus by about 150-fold at 9 dpi, when co-treated with compounds KPT-330 or KPT-335. These two drugs are CRM1 inhibitors. Green: HBc. Red: NTCP. Blue: DAPI. FIG. 2B shows that quantitative results of the IFA assay in FIG. 2A are summarized in a bar graph. ***p<0.001. FIG. 2C shows an outline of a time course kinetic experiment of drug treatment using an HBV infection system. Briefly, the infected culture of HepG2-NTCP-AS cells are treated with KPT-335 (0.5-1 μM) at 0, 3, and 5 dpi continuously until cell harvesting. Media are collected for ELISA of HBsAg and HBeAg. Cells are harvested for further analysis by Southern blot (SB) and Northern blots (NB). FIG. 2D shows that only earlier co-treatment can inhibit HBsAg and HBeAg in the medium by ELISA. FIGS. 2E and 2F show that SINE compounds strongly inhibit HBV DNA synthesis and cytoplasmic capsid-associated pgRNA in the infection assay by Southern and Northern blot analyses. The level of total cytoplasmic viral RNA is reduced moderately by treatment with SINE compounds (1 μM). Comparisons of relaxed circular (RC) DNA banding intensities are as described in FIGS. 1A-1B legend, and normalized here to the no drug treatment control. SS: single-strand RNA.

FIGS. 3A to 3C show summaries of the nuclear export signals of HBc and models of pgRNA encapsidation occurring in the cytoplasm versus nucleus. FIG. 3A shows HBc capsids associated with CRM1 and Nup358 for nuclear export through nuclear pores. The spikes of HBc capsid particles are highlighted in a cryoEM 3D reconstruction density map. HBc protein contains two distinct nuclear export domains mediated by the CRM1 and the NXF1 pathways, respectively. The NES^{CRM1} motif is located near the tip of the spike of HBc 52-95. The NES^{NXF1} motifs are located in the arginine-rich RNA-binding domain near the C-terminus of HBc 147-183. The encapsidated pgRNA (10-20%) can be exported by the CRM1-mediated machinery. In contrast, non-encapsidated pgRNA (80%) can be exported by the NXF1-mediated machinery. HBc capsids associate with CRM1 before nuclear export through the nuclear pore complex. Nup358: a nucleoporin involved in nuclear export. FIG. 3B shows that the old dogma of cytoplasmic initiation of viral RNA encapsidation. The present invention proposes here that this cytoplasmic event can occur only later in HBV life cycle. FIG. 3C shows that an alternative new model proposes that the encapsidation of viral RNAs can also be initiated de novo in the nucleus at the earlier time point post viral entry. Such immature capsids assembled in the nucleus are rapidly exported to the cytoplasm for further genome maturation. Cytoplasmic mature capsids containing relaxed circle (RC) DNA can recycle back to the nucleus, and amplify the covalently closed circular cccDNA pool via repetitive nucleocytoplasmic shuttling of nucleocapsids. This nucleus-initiation model for encapsidation of pgRNA, at a lower concentration earlier in HBV life cycle, is not mutually exclusive with the cytoplasm-initiation model occurring later in HBV life cycle. θ: CRM1 inhibitors. Pol: polymerase.

FIG. 4 shows that knockdown of CRM1 by siRNA treatment significantly increases the nuclear HBc protein level. The knockdown efficacies are shown in the left panel. A wild type HBV genome and siRNAs are co-transfected into HuH-7 cells. Quantitation results of the IFA assay are plotted in the right panel. A combination treatment with both CRM1 and NXF1 specific siRNAs results in further accumulation of nuclear HBc. p value: **<0.01; ***<0.001. The siRNAs of TAP/NXF1 gene (ON-TARGETplus SMARTpool) are purchased from Dharmacon (USA). The siRNAs HSS111415, HSS111416, and HSS111417 are specific for CRM1/XPO1 gene and are purchased from Invitrogen (USA). SiNonTarget is used as a negative control in the siRNA knockdown experiment.

FIG. 5 shows that hepatitis B virus secretes a total of six different viral and subviral particles in hepatocyte cell culture. A cartoon illustration of secreted HBV virions, naked capsids, and HBsAg particles. Left panel: genome-containing and genome-free empty virions contain both surface antigen and core proteins; middle panel: genome-containing and genome-free naked capsids contain no surface antigens; right panel: HBV surface antigen particles contain no core protein. Only the genome-containing virions are infectious.

FIGS. 6A and 6B show a cartoon illustration for the analysis methods of extracellular and intracellular HBV viral and subviral particles. FIG. 6A shows that the extracellular HBV particles in the media are first precipitated by ultracentrifugation through a sucrose cushion before native agarose gel electrophoresis. Viral and subviral particles are separated and characterized by Southern and Western blot analyses. Anti-HBc and anti-HBs antibodies are used consecutively for the same Western blot filters. Southern blot is performed in a separate agarose gel which allows the differentiation of genome-containing (G⁺) from genome-free (G⁻) viral and subviral particles. FIG. 6B shows that the intracellular core-associated viral RNA and DNA are purified by PEG precipitation and nucleic acid extraction. Then, the RNA/DNA signals are analyzed by Northern blot and Southern blot. 3.5 kb pgRNA: full-length 3.5 kb pre-genomic RNA. RT RNA: reverse transcripted RNA.

FIGS. 7A to 7C show that CRM1 inhibitors block secretion of HBV virions but not naked capsids. FIG. 7A shows that secretions of WT-HBV virions and HBsAg particles are strongly inhibited by SINE compound at 0.1 μM in a dose-response curve. While virion secretion is clearly reduced when SINE compound concentration is increased, secretion of naked capsid is far less sensitive to drug treatment. Similarly, Southern blot analysis reveals that the secretion of genome-containing virions is much more sensitive to SINE compounds than the secretion of genome-containing naked capsids. FIG. 7B shows that as a control to the extracellular secretion profiles in FIG. 7A, the present invention examines the intracellular WT-HBV capsid protein, capsid-associated viral RNA and DNA in the same dose-response experiment by the SINE compound treatment. FIG. 7C shows that SINE compound treatment also inhibits genome-free empty virion secretion of a replication-defective HBc NES mutant L65A/V89A. This mutant secretes no naked capsids. V/NC: Virions/naked capsids.

FIG. 8 shows that cell proliferation is monitored by alamarBlue and CCK-8 assays after treatment with CRM1 inhibitors. No significant anti-proliferation effect is detected below 0.25 μM SINE compound.

FIG. 9 shows that CRM1 is directly involved in HBV virion secretion. In an HBV transient transfection system in HuH-7 cells, Lentivirus shCRM1 #1 and #2 significantly knocked down CRM1 expression and reduced HBV virion secretion. The bar graph (right panel) is based on the signal intensities of virions and naked capsids (left panel) using densitometry and Image J. The mean values are calculated from at least three independent experiments. shScramble: a non-specific control. The vertical dotted line indicates splicing of data from the same gel. HBc protein signals of secreted virions (solid bar) and naked capsids (open bar) in the shScramble experiment, are shown here as a 100% reference control. Lentivirus shRNAs for CRM1 knock down (ShCRM1: TRCN0000152787, TRCN0000150975, TRCN0000338401, and TRC1.Scramble) are purchased from RNAiCore, Academia Sinica, Taiwan.

FIGS. 10A and 10B show two different models of HBV virion secretion. FIG. 10A shows a current model for virion secretion which postulates the direct budding of cytoplasmic capsids into ER/Golgi in a nucleus-independent manner. FIG. 10B shows that in the new model of the present invention, CRM1-mediated nuclear export of HBc capsids is followed immediately by the microtubule-mediated transport from MTOC (microtubule organization center) at nuclear pore to perinuclear ER/Golgi for envelopment and virion egress. Both genome-containing and genome-free capsids can undergo nucleocytoplasmic shuttling. Nu: nucleus. Cy: cytoplasm. ER: endoplasmic reticulum. Golgi: Golgi apparatus.

FIG. 11 shows that KPT-8602 at 0.1 μM blocks HBV virion secretion. An HBV replicon plasmid is transfected into HuH-7 human hepatoma cell line. SINE compound KPT-8602 is added to the transfected culture at 0 hr post-transfection (i.e., co-treatment). Extracellular media are collected on day 5 (120 hr) post-transfection. Assays for virion secretion are as described in FIG. 6A. Secretion of naked capsids is 100-fold (10 μM/0.1 μM) less sensitive to KPT-8602 treatment than virions.

FIG. 12 shows that KPT-8602 is more inhibitory than KPT-335 for HBV virion secretion in a broad range of concentrations at 0.1, 0.25, 0.5 and 1 μM. In contrast to virions, secretion of naked capsids is much less sensitive to KPT-335 or KPT-8602.

### DETAILED DESCRIPTION OF THE INVENTION

The term “a” or “an” as used herein is to describe elements and ingredients of the present invention. The term is used only for convenience and providing the basic concepts of the present invention. Furthermore, the description should be understood as comprising one or at least one, and unless otherwise explicitly indicated by the context, singular terms include pluralities and plural terms include the singular. When used in conjunction with the word “comprising” in a claim, the term “a” or “an” may mean one or more than one.

The term “or” as used herein may mean “and/or.”

The present invention provides a method for treating symptoms of a disease associated with an infection caused by hepatitis B virus (HBV) in a subject, comprising administering to the subject suffering from HBV infection a composition comprising a therapeutically effective amount of a CRM1 inhibitor.

The present invention also provides a use of a composition for the manufacture of a drug for treating symptoms of a disease associated with an infection caused by hepatitis B virus (HBV), wherein the composition comprises a therapeutically effective amount of a CRM1 inhibitor.

As used herein, the term “hepatitis B virus” or “HBV” is meant any hepatitis B virus of any serotype or genotype. In some embodiments, the HBV is any one of genotypes A-J.

In another embodiment, the disease associated with the HBV infection comprises chronic liver disease or disorder, liver inflammation, liver fibrotic condition, proliferative hepatocellular disorder, hepatocellular carcinoma, hepatitis D virus co-infection, acute HBV infection, chronic hepatitis B or chronic HBV infection. Therefore, the CRM1 inhibitor is able to treat the diseases caused by the HBV infection.

As used herein, the term “treating” refers to prophylactic or therapeutic treatments, wherein the subject is to prevent, reverse, alleviate, ameliorate, inhibit, slow down or stop the progression or severity of a condition associated with the HBV infection.

In one embodiment, the subject is an animal, preferably a mammal, more preferably a human. In another embodiment, the subject is a subject with chronic HBV infection.

In some aspects, CRM1 (chromosome region maintenance 1), also known as exportin 1 (XPO1), is a major export receptor for nuclear proteins containing nuclear export signals (NESs) with clustering hydrophobic residues. As used herein, the term “CRM1 inhibitor” refers to compounds that either inhibit the production, generation, synthesis, processing, or modification, of CRM1 mRNA of CRM1 mRNA, that facilitate the degradation or sequester the CRM1 mRNA, that inhibit the translation from CRM1 mRNA into protein, or that inhibit the half-life (longevity, turnover) or biological the activity of CRM1 protein. Non-limiting examples of the CRM1 inhibitors include Leptomycin A, Leptomycin B, Leptomycin analogs, an RNA that interferes with CRM1 expression or mRNA thereof, ratjadone, valtrate, acetoxychavicol acetate, an oral CRM1 inhibitor (CBS9106), a selective inhibitor of nuclear export (SINE), a natural compound that inhibits CRM1, or natural products such as curcumin.

In some embodiments, the SINE compounds are drugs that block CRM1 binding to the cargo. Therefore, the SINE compounds can inhibit CRM1-dependent HBV virion secretion. In some aspects, the SINE compounds comprise KPT-330, KPT-8602, KPT-185, KPT-276, or KPT-335.

In another embodiment, the CRM1 inhibitor comprises an interfering RNA molecule, KPT-330, KPT-8602, KPT-185, KPT-276, KPT-335, or derivatives thereof. In some aspects, the interfering RNA molecule has the function to interfere with CRM1 mRNA expression or stability thereof. In one embodiment, the interfering RNA molecule comprises short hairpin RNA (shRNA), small interfering RNA (siRNA), antisense RNA, oligonucleotides or microRNA (miRNA).

In the present invention, the nuclear export of HBV pgRNA-containing capsids is facilitated by the CRM1-mediated pathway in the hepatocytes. Therefore, the CRM1 inhibitor can inhibit the nuclear export of encapsidated HBV pgRNA and HBV pgRNA-containing capsids. Furthermore, the CRM1 inhibitor is able to inhibit HBV virion secretion. In one embodiment, the CRM1 inhibitor treats the HBV infection by inhibiting a nuclear export pathway of HBV core particles. In a preferred embodiment, the CRM1 inhibitor treats the HBV infection by inhibiting a nuclear export of HBV capsids containing mature relaxed circle (RC) DNA genome.

In the present invention, the CRM1 inhibitor and other anti-HBV drug can be used in combination to treat the HBV infection. In another embodiment, the composition further comprises an anti-HBV drug. In addition, the active ingredients of the anti-HBV drug are different from that of the CRM1 inhibitor. In another embodiment, the anti-HBV drug comprises HBV polymerase inhibitor, HBV immunomodulator, cytokines or interferons. In some embodiments, the other anti-HBV drugs comprise lamivudine, telbivudine, tenofovir, entecavir, adefovir dipivoxil, alfaferone, alloferon, celmoleukin, clevudine, emtricitabine, famciclovir, hepatect CP, interferon-1a, interferon-1b, interferon-2a, interferon-2b, interleukin-2, mivotilate, nitazoxanide, peginterferon alfa-2a, ribavirin, roferon-A, sizofiran, ampligen, phosphazid, heplisav, levamisole or propagermanium.

In some embodiments, the forms of the anti-HBV drug comprise pegylated or non-pegylated interferon, immune modulators, therapeutic vaccines, HBV protein-specific monoclonal antibodies, nucleos(t)ide analogs, anti-sense molecules, siRNA, entry inhibitors, capsid inhibitors, HBsAg inhibitors, HBx inhibitors, PDL1 inhibitors, other checkpoint inhibitors, FXR agonists, apoptosis inducer, gene editing molecules. In a preferred embodiment, the nucleos(t)ide analogs of the anti-HBV drug comprise Epivir, Adefovir dipivoxil, Entecavir, Telbivudine, Tenofovir, Cledvudine, or their prodrugs, or their derivatives.

In some aspects, the CRM1 inhibitor and other anti-HBV drug can form a drug combination for the treatment of hepatitis B virus infection. The CRM1 inhibitor and the anti-HBV drug as independent entities (e.g., pharmaceutical compositions, pharmaceutical formulations) can be administered simultaneously, in parallel or sequentially, and without specific time limits to the subject, wherein the active ingredients of the composition administered to the subject reaches a therapeutically effective amount. Therefore, the active ingredients in the drug combination of the present invention may be administered individually, or partially or in whole thereof. The active ingredients in the drug combination of the present invention may be administered substantially at different times, or substantially simultaneously administered in part or in whole thereof.

In the present invention, the CRM1 inhibitor is advantageously formulated in a pharmaceutical composition, together with a pharmaceutically acceptable carrier.

As used herein, the term “pharmaceutically” or “pharmaceutically acceptable” refers to molecular entities and compositions that do not produce an adverse, allergic or other untoward reaction when administered to a mammal, especially a human, as appropriate. A pharmaceutically acceptable carrier, excipient or diluent refers to a non-toxic solid, semi-solid or liquid filler, diluent, encapsulating material or formulation auxiliary of any type.

The pharmaceutically acceptable carriers and excipient that may be used in the compositions of this invention include, but are not limited to, ion exchangers, alumina, aluminium stearate, lecithin, self-emulsifying drug delivery systems (SEDDS) such as d-a-tocopherol polyethyleneglycol 1000 succinate, surfactants used in pharmaceutical dosage forms such as Tweens or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

As appreciated by skilled artisans, the pharmaceutical composition is suitably formulated to be compatible with the intended route of administration. Examples of suitable routes of administration include topical route, oral route, intranasal route, intraocular route, parenteral route, and including intramuscular, subcutaneous, intravenous, intraperitoneal or local injections. The oral route can be used, provided that the composition is in a form suitable for oral administration, i.e. able to protect the active principle from the gastric and intestinal enzymes. Preferably the CRM1 inhibitor for the use according to the invention is administered by topical route, oral route, intranasal route, intraocular route, parenteral route, or by intramuscular, subcutaneous, intravenous, intraperitoneal or local injections.

Preferably, the pharmaceutical composition contains carriers that are pharmaceutically acceptable for an injectable formulation. They may in particular be sterile, isotonic, saline solutions (monosodium phosphate, disodium phosphate, sodium chloride, potassium chloride, calcium chloride or magnesium chloride etc., or mixtures of such salts), or dry, in particular lyophilized, compositions which by means of the addition, as appropriate, of sterilized water or physiological saline, can form injectable solutes.

The term “therapeutically effective amount” used herein is a therapeutic dose which can prevent, decrease, stop or reverse a symptom developed in a subject under specific conditions, or partially, completely alleviates symptoms already exist under specific conditions when the subject begins receiving the treatment.

The doses used for the administration can be adapted as a function of various parameters, and in particular as a function of the mode of administration used, of the relevant pathology, or alternatively of the desired duration of treatment. For example, it is well within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Preferably, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the subject to be treated. A medicament typically contains from about 0.01 mg to about 500 mg of the active ingredient, preferably from 0.01 mg to about 100 mg of the active ingredient. An effective amount of the drug is ordinarily supplied at a dosage level from 0.01 mg/kg to about 100 mg/kg of body weight per day, especially from about 0.1 mg/kg to 50 mg/kg of body weight per day.

In one embodiment, the therapeutically effective amount of the CRM1 inhibitor ranges from 0.01 mg/kg to 100 mg/kg. In a preferred embodiment, the therapeutically effective amount of the CRM1 inhibitor ranges from 0.1 mg/kg to 50 mg/kg. In a more preferred embodiment, the therapeutically effective amount of the CRM1 inhibitor ranges from 0.2 mg/kg to 25 mg/kg.

In one embodiment, the therapeutically effective amount is administered in a single dose per day. In a preferred embodiment, the therapeutically effective amount is administered in two or more doses per day.

The composition of the present invention may be administered on a routine schedule. As used herein a routine schedule refers to a predetermined designated period of time. The routine schedule may encompass periods of time which are identical or which differ in length, as long as the schedule is predetermined. For instance, the routine schedule may involve administration one dose a day, every day, every two days, every three days, every four days, every five days, every six days, a weekly basis, a monthly basis or any set number of days or weeks there-between. Alternatively, the predetermined routine schedule may involve administration on a one daily basis for the first week, followed by a daily basis for several months, etc. In other embodiments, the present invention provides that the drug(s) may be taken orally and that the timing of which is or is not dependent upon food intake. Thus, for example, the drug can be taken every morning and/or every evening, regardless of when the subject has eaten or will eat. In one embodiment, the composition is administered every week. In a preferred embodiment, the administration of the composition continues for a period that extends for at or about or longer than 3-12 months. In another embodiment, the composition is administered for at least 3 months. In a preferred embodiment, the composition is administered for at least 6 months. In a preferred embodiment, the composition is administered for at least 12 months.

The present invention further provides a method of inhibiting a nuclear export pathway of HBV core particles in an in vitro cell, comprising administering a composition comprising a CRM1 inhibitor into the cell with HBV infection or transfection.

The present invention also provides a use of a composition for the manufacture of a drug for inhibiting a nuclear export pathway of HBV core particles in an in vitro cell, wherein the composition comprises a therapeutically effective amount of a CRM1 inhibitor.

In one embodiment, the concentration of the CRM1 inhibitor for treating the cell ranges from 0.1 to 10 μM. In a preferred embodiment, the concentration of the CRM1 inhibitor for treating the cell ranges from 0.3 to 5 μM. In a more preferred embodiment, the concentration of the CRM1 inhibitor for treating the cell ranges from 0.5 to 3 μM.

Thus, the present invention demonstrates the CRM1 inhibitor blocks the nuclear export of HBV RNA-containing capsids or HBV virion secretion. Therefore, for the subject with chronic established HBV infection, long-term treatment, rather than short term treatment, with CRM1 inhibitors is needed to prevent reinfection of uninfected fresh hepatocytes from liver regeneration. In conclusion, CRM1 inhibitors are potential candidates for the treatment of HBV infection.

### EXAMPLES

The embodiment of the present invention could be implemented with different content and is not limited to the examples described in the following text. The following examples are merely representative of various aspects and features of the present invention.

### Part I

The present invention taught an effective method of using CRM1 inhibitors to block the transport of HBV encapsidated pregenomic RNA (pgRNA) from nucleus to cytoplasm. The drug target here was an early event in HBV life cycle.

The present invention identified CRM1-specific nuclear export signals (NES^{CRM1}) clustering at the tip of the spike of HBc protein particles (capsids). Mutagenesis of NES^{CRM1} arrested HBc capsids in the nucleus.

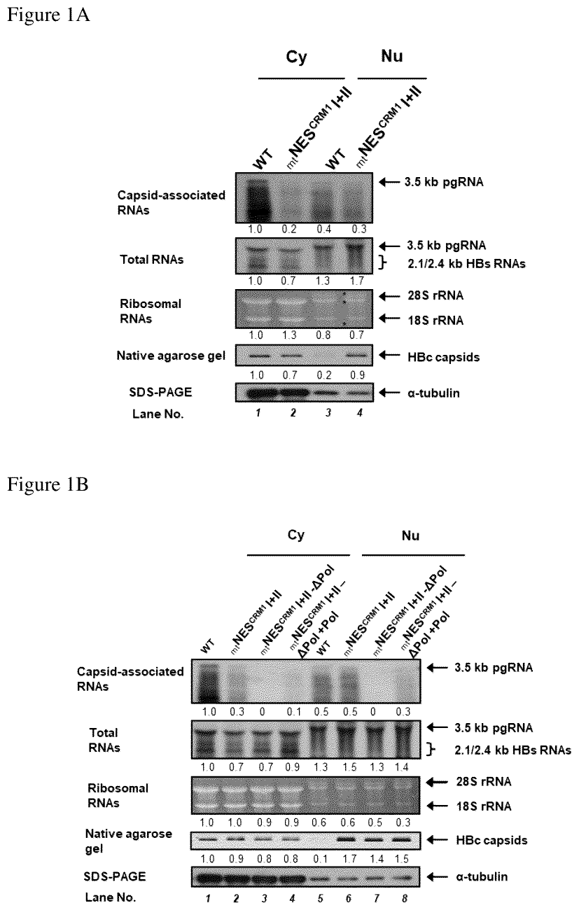

Since NES^{CRM1} mutant arrested HBc capsids in the nucleus, the present invention asked if these capsids contain any HBV-specific RNA or DNA. In the upper panel of FIG. 1A, WT contained much stronger signal of capsid-associated RNA in the cytoplasm than in the nucleus (lane 1»lane 3). However, the opposite was observed in the NES mutant (lane 2<lane 4). This result suggested that 1) the nuclear pgRNA signal of NES mutant was not a contamination from the cytoplasmic pgRNA fraction; and 2) it was likely to originate from encapsidated pgRNA arrested in the nucleus. Another way to analyze the data in FIG. 1A, top panel, was to compare capsid-associated RNA in lane 1 vs. lane 2 (5-fold difference) as well as lane 3 vs. lane 4 (1.3-fold difference). Again, the large difference in the encapsidated pgRNA level in the cytoplasm between WT and NES mutant was not paralleled by their minor difference in the nucleus. These results in FIG. 1A were reproduced faithfully in the top panel of FIG. 1B. Taken together, NES^{CRM1} on the spike tip of HBV capsid particles was required for nuclear export of HBV-RNA containing capsids.

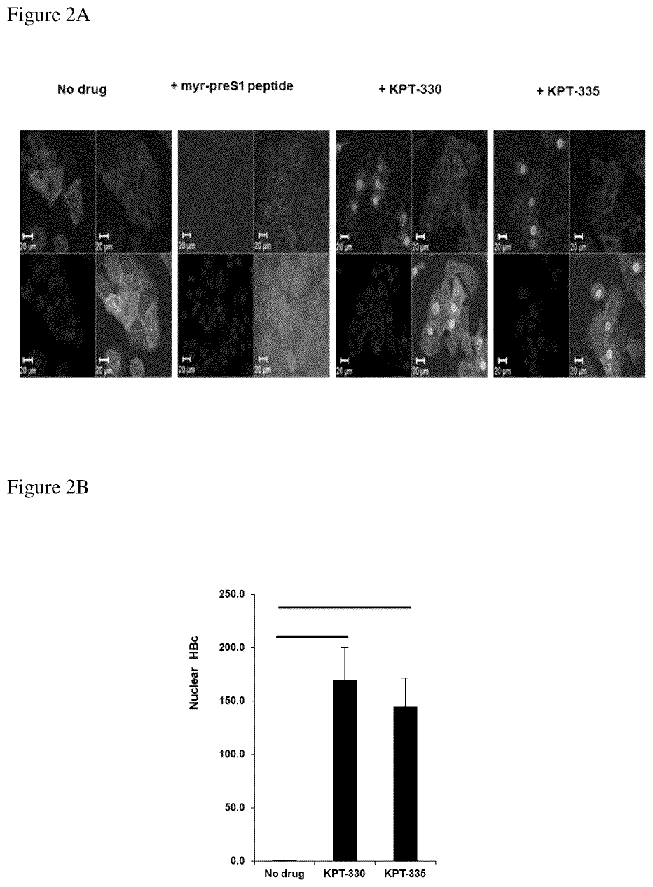

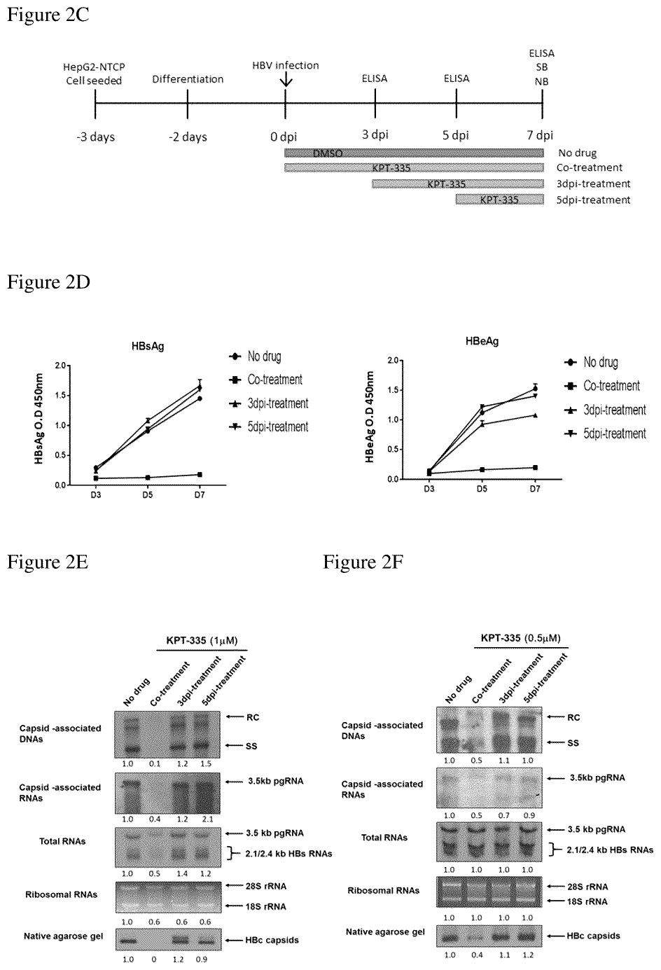

The results in FIGS. 1A-1B by genetic mutant analysis was also supported by the results from the CRM1 drug treatment experiment (FIGS. 2A-2F). KPT-330 and KPT-335 are known CRM1 inhibitors. In an HBV in vitro infection system, when KPT-330 or KPT-335 were added to the cell culture before or soon after the infection with HBV, HBc was strongly arrested in the nucleus by immunofluorescence microscopy (FIG. 2A). Relative to the control with no drug treatment, KPT-330 and KPT-335 could increase nuclear HBc by approximately 150-fold (FIG. 2B). HBsAg and HBeAg were HBV serum markers. In a kinetic experiment, drug added at 3 or 5 dpi had no apparent effect on HBV, as measured by ELISA for HBsAg and HBeAg in the medium. However, when drug and virus were added at the same time (co-treatment), complete inhibition of HBsAg and HBeAg expression was observed by ELISA (FIGS. 2C and 2D). Furthermore, cotreatment with KPT-335 at 0.5-1 μM concentration strongly inhibited HBV RNA encapsidation, DNA synthesis and formation of HBc capsids (FIGS. 2E and 2F).

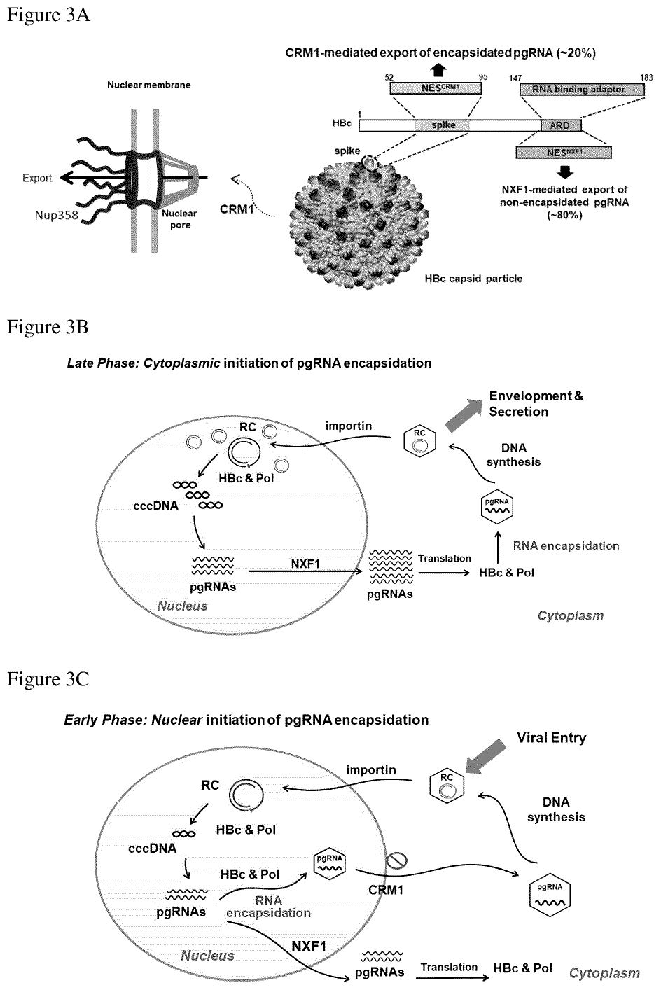

As outlined in the cartoon of FIG. 3A, CRM1 machinery could recognize the NES^{CRM1} motif at the spike tip, and thus facilitated the nuclear export of HBc capsid particles through the nuclear pore. The results in FIGS. 1A-1B and 2A-2F led the present invention to propose a new model of HBV life cycle (FIG. 3B). In this new model, pgRNA encapsidation could occur in the nucleus as an early event, and de novo assembled RNA-containing capsids needed to be exported by CRM1 to the cytoplasm for reverse transcription and DNA synthesis. In contrast, in the old model (FIG. 3C), pgRNA encapsidation occurred mainly in the cytoplasm. If so, CRM1 inhibitors (KPT-335) should have no effect on the HBV life cycle. Because KPT-335 had no effect on HBV when added at 3 or 5 dpi, it suggested that cytoplasmic initiation of pgRNA encapsidation could take place only later in the HBV life cycle (3 or 5 dpi).

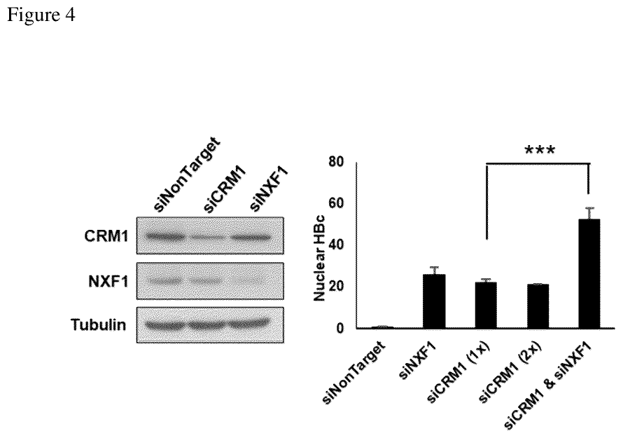

In addition to SINE compounds, such as KPT-335, CRM1 can also be inhibited by the siRNA treatment. In FIG. 4, a nearly 20-fold increase in nuclear HBc was observed, when CRM1 or NXF1 was individually inhibited by their respective siRNAs. A combinatory siRNA treatment resulted in an additive effect (around 50-fold increase) on nuclear HBc (FIG. 4). This siRNA experiment demonstrated that CRM1-specific siRNA can reduce CRM1 protein expression leading to the increased accumulation of HBc protein in the nucleus. In the past two years (2021-present), delivery of SARS-CoV-2 RNA vaccine has been successful by using lipid-capsulated nanoparticles. It is conceivable that CRM1-specific interfering RNAs (siRNA, shRNA) could be delivered in a similar manner for treating hepatitis B patients.

### Part II

The present invention taught an effective method of using CRM1 inhibitors to block the secretion (egress, release) of HBV virions. The drug target here was a very late event in HBV life cycle.

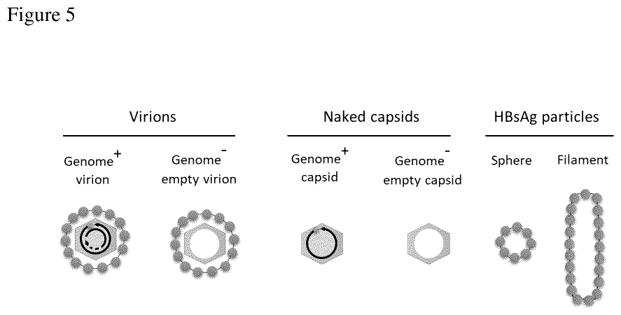

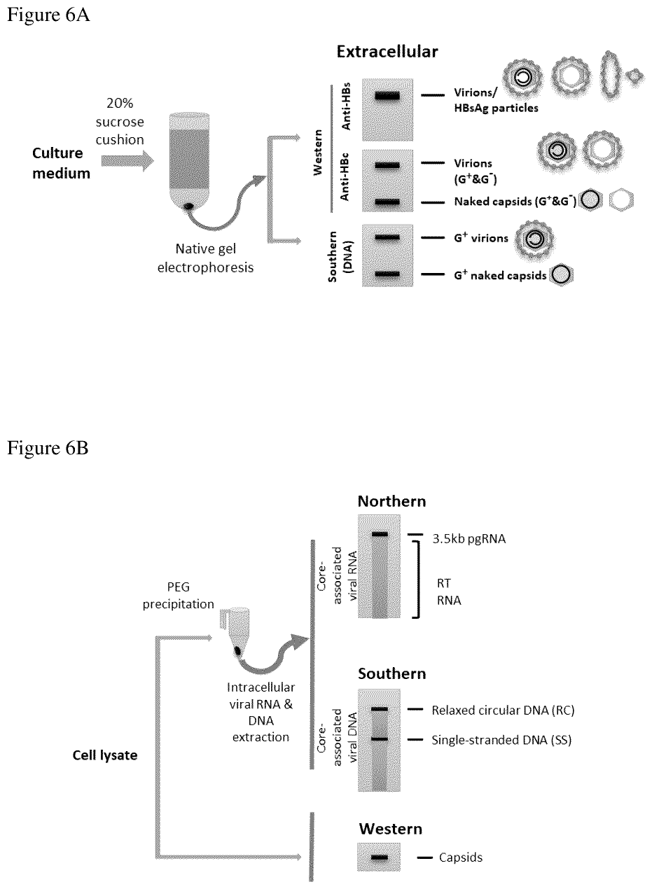

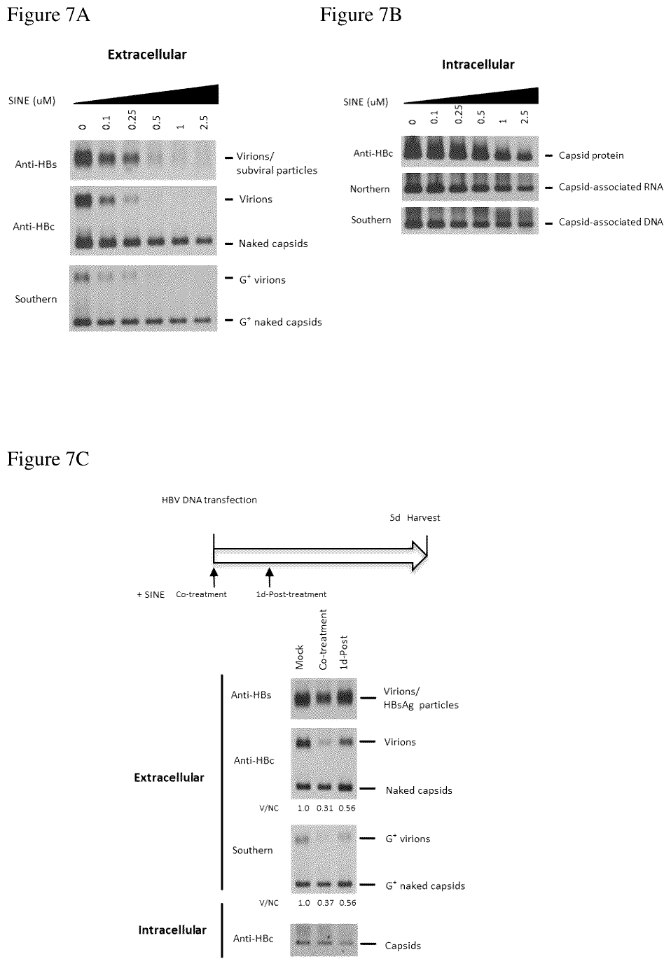

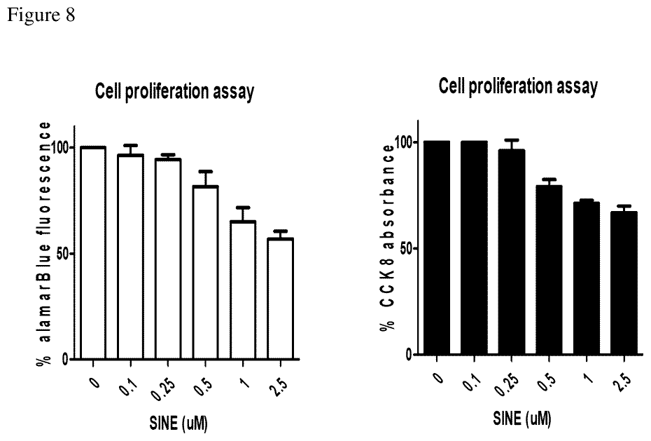

The present invention would present another novel finding that CRM1 inhibitors could not only inhibit nuclear export of hepatitis B virus RNA-containing capsids, but also block virion release. HBV secreted a total of six different viral and subviral particles in hepatocyte cell culture (FIG. 5). The only infectious particles were the genome-containing virions, which contained envelope protein, capsids, and encapsidated relaxed circle DNA genome. In FIGS. 6A and 6B, a cartoon illustrated the analysis methods of extracellular and intracellular HBV viral and subviral particles. In FIG. 7A, it was a big surprise that SINE compounds (KPT-335) could almost completely block virion secretion at 0.5 μM. At this concentration, there was no significant effect on capsid protein and capsid-associated RNA (RNA encapsidation) (FIG. 7B). Again, co-treatment by adding virus and KPT simultaneously was most effective against virion secretion. Adding drug at 1-day post infection was not effective in blocking virion secretion (FIG. 7C). The potential cytotoxicity of KPT-335 was evaluated using two different methods—Alamar Blue and CCK-8 assays (FIG. 8). No significant anti-proliferation effect on HuH-7 cells was detected below 0.25 μM SINE compound.

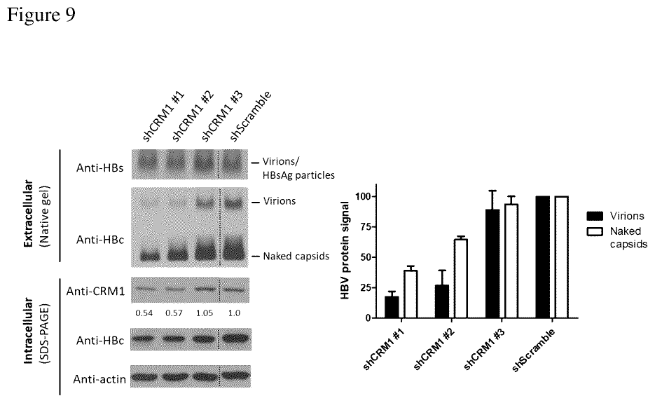

In addition to SINE compounds, another method to reduce CRM1 activity is by using the CRM1-specific interfering RNA, such as siRNA or shRNA. As shown in FIG. 9, CRM1-specific shRNAs, shCRM1 #1 and shCRM1 #2, significantly decrease the intracellular level of CRM1 protein and the extracellular level of secreted visions. As discussed earlier in FIG. 4, delivery of SARS-CoV-2 RNA vaccine has been successfully demonstrated by Moderna or BNT-Pfizer vaccine companies using lipid-capsulated nanoparticles. It is conceivable that CRM1-specific interfering RNAs (siRNA, shRNA) can be delivered in a similar manner for treating hepatitis B patients (FIGS. 4 and 9).

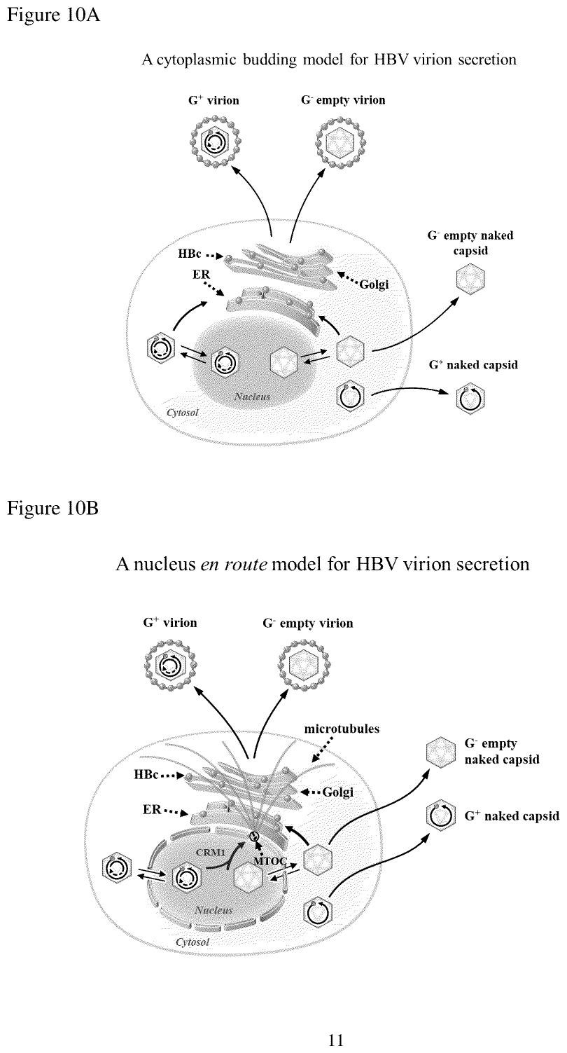

In the old concept of the present invention (FIG. 10A), HBV capsids containing mature genome of relaxed circle (RC) DNA, could be secreted to the extracellular compartment by direct budding from cytoplasm into the ER/Golgi. However, the results in FIGS. 5, 6A-6B, 7A-7C and 8-9 led to a new “nucleus en route model” for HBV virion secretion (FIG. 10B). In this model, the bulky icosahedral particles (T=3 or 4) of HBV mature capsids in the cytoplasm could not directly bud into ER/Golgi for envelopment and egress. Rather, mature capsids needed to enter the nucleus first before reaching the perinuclear ER/Golgi for virion secretion. CRM1-mediated nuclear export was therefore a crucial step in HBV virion secretion.

### Part III

The present invention presented new data of improved efficacy of a second-generation SINE compound KPT-8602 (Eltanexor). Based on Part I and Part II, the present invention invented a 2-bird-1-stone therapeutic method for hepatitis B patients.

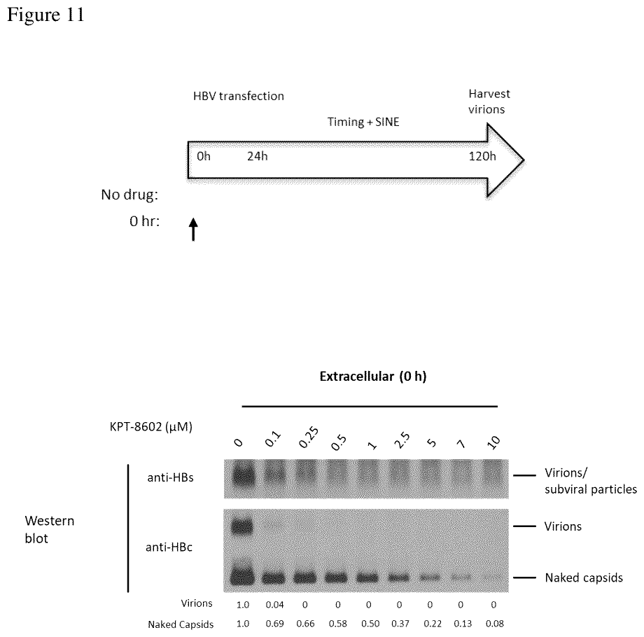

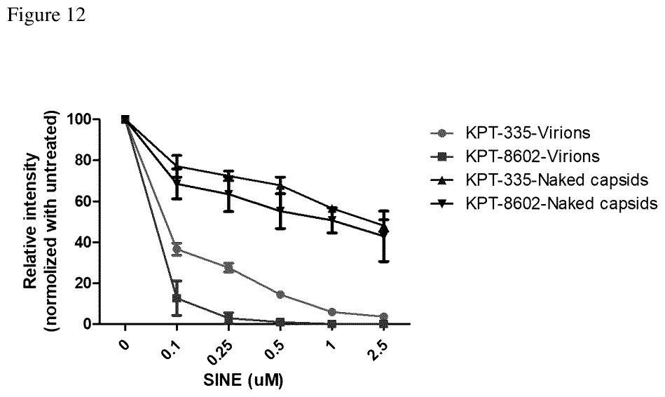

Recently, a second-generation SINE compound, KPT-8602 (Eltanexor), is commercially available. KPT-8602 is structurally related to KPT-335 (Verdinexor) and is being tested in clinical trials (e.g., ClinicalTrials.gov Identifier: NCT02649790). Using a HuH-7 hepatoma cell line transfected with an HBV replicon plasmid, the present invention found that earlier co-treatment of KPT-8602 at 0.1 μM could nearly completely block HBV virion secretion (FIG. 11). Side-by-side comparisons between KPT-8602 and KPT-335 indicated that KPT-8602 was more potent and less cytotoxic than KPT-335 (FIG. 12).

### Conclusion

In both infection (FIGS. 2C-2F) and transfection systems (FIGS. 7C and 11), earlier co-treatment with SINE compounds was the key to successful inhibition of HBV gene expression, RNA encapsidation, DNA synthesis, and virion secretion. When drug was added to the HBV cell culture at a later time point (1, 3, or 5 dpi) post-infection or-transfection, resistance to SINE compounds was observed, resulting in no significant antiviral effect. Therefore, for the vast majority of hepatocytes already infected with HBV in the liver of chronic hepatitis B patients, “short term” treatment with SINE compounds may not have any efficacy in viral clearance. In contrast, in “long term” treatment, the subpopulation of naïve hepatocytes with no HBV infection would be generated from the metabolic turnover of the regenerating liver. A susceptibility window for earlier co-treatment could then be targeted by SINE compounds in a long term treatment regimen.

Those skilled in the art recognize the foregoing outline as a description of the method for communicating hosted application information. The skilled artisan will recognize that these are illustrative only and that many equivalents are possible.

1. A method for treating symptoms of a disease associated with an infection caused by hepatitis B virus (HBV), which comprises administering a composition containing a therapeutically effective amount of a CRM1 inhibitor into an HBV-infected subject, wherein the CRM1 inhibitor prevents uninfected and freshly regenerated hepatocytes from being infected in the HBV-infected subject by inhibiting a nuclear export pathway of HBV core particles and by blocking HBV virion secretion.

2. The method of claim 1, wherein the disease associated with the HBV infection comprises chronic liver disease or disorder, liver inflammation, liver fibrotic condition, proliferative hepatocellular disorder, hepatocellular carcinoma, hepatitis D virus co-infection, acute HBV infection, chronic hepatitis B or chronic HBV infection.

3. The method of claim 1, wherein the CRM1 inhibitor comprises an interfering RNA molecule, KPT-330, KPT-8602, KPT-185, KPT-276, KPT-335, or derivatives thereof.

4. The method of claim 3, wherein the interfering RNA molecule comprises shRNA, siRNA, antisense RNA, oligonucleotides or miRNA.

5. (canceled)

6. The method of claim 1, wherein the composition further comprises an anti-HBV drug.

7. The method of claim 6, wherein the anti-HBV drug comprises HBV polymerase inhibitor, HBV immunomodulator, cytokines or interferons.

8. The method of claim 1, wherein the therapeutically effective amount of the CRM1 inhibitor ranges from 0.01 mg/kg to 100 mg/kg.

9. The method of claim 1, wherein the composition is administered every week.

10. The method of claim 1, wherein the composition is administered for at least 3 months.
